(19) United States
(12) Patent Application Publication
(10) Pub. No.: US 2010/0178333 A1
(43) Pub. Date: Jul. 15, 2010

(54) SUSTAINED RELEASE DOSAGE FORM OF PHENOTHIAZINE DERIVATIVES CONTAINING CHANNELIZER

(75) Inventors: Jayanta Kumar Mandal, Gujarat (IN); Nitesh Nalinchandra Pandya, Gujarat (IN); Kirti Bansidhar Maheshwari, Gujarat (IN)

Correspondence Address: HAMRE, SCHUMANN, MUELLER & LARSON, P.C., P.O. BOX 2902, MINNEAPOLIS, MN, 55402-0902, US

(73) Assignee: ASTRON RESEARCH LIMITED, Ahmedabad, Gujarat (IN)

(21) Appl. No.: 12/063,812

(22) PCT Filed: Jan. 23, 2007
(86) PCT No.: PCT/IN07/00031

§ 371 Date: Feb. 14, 2008

(30) Foreign Application Priority Data
Jan. 25, 2006 (IN) 124/MUM/2006

(51) Int. Cl. A61K 9/52; A61K 9/14; A61K 9/22; A61K 31/554; A61P 25/18

(52) U.S. Cl. 424/452; 424/490; 424/465; 514/211.13

(57) ABSTRACT

Once a day sustained release solid oral dosage form of phenothiazine derivative preferably the dibenzothiazepine derivative and their pharmaceutically acceptable salts comprising of a channelizer, rate controlling polymer and suitable pharmaceutically acceptable excipients. The formulation of the present invention is in the form of tablet or capsule which provides a sustained drug action upto 24 hours upon single dose administration.

### FIELD OF THE INVENTION

The present invention relates to novel solid oral pharmaceutical formulation, more particularly sustained release formulation of antipsychotic drug like phenothiazine derivative. The preferable phenothiazine derivatives in the formulation of the present invention are heterocyclic analogue of phenothiazines, more preferably dibenzazepine derivative and most preferably the dibenzothiazepine compounds like quetiapine and pharmaceutically acceptable salt thereof, along with a channelizer which facilitates the release of active pharmaceutical ingredient from the dosage form. This dosage form is a solid oral dosage form which maintains an effective plasma drug concentration over an extended period of time and thereby provides a sustained drug action.

### BACKGROUND

Antipsychotic drugs are primarily used to treat psychotropic disorders and other mental and emotional conditions.

Phenothiazines are a class of antipsychotic drugs used for the treatment of serious mental and emotional disorders, including schizophrenia.

Phenothiazine derivatives include, aliphatic and piperidine phenothiazines (e.g. chlorpromazine, triflupromazine, promazine, mesoridazine, perphenazine etc.), piperazine phenothiazines (e.g. prochlorperazine, fluphenazine, trifluperazine etc.) and heterocyclic analogues of phenothiazines.

Heterocyclic analogue of phenothiazines include thioxanthenes (e.g. thiothixene), arylbutylpiperidines (including fluorobutyrophenone e.g. haloperidol, and diarylbutylpiperidine e.g. pimozide), dihydroindolones (e.g. molindone hydrochloride), benzisoxazoles (e.g. risperidone) and dibenzazepines.

The dibenzazepines includes dibenzoxazepine (e.g. loxapine), dibenzodiazepine (e.g. clozapine), and dibenzothiazepine (e.g. quetiapine).

U.S. Pat. No. 4,879,288 describes the compound 11-[4-[2-(2-hydroxyethoxy)ethyl]-1-piperazinyl]dibenzo [b,f][1,4]thiazepine as a novel antipsychotic drug of class dibenzothiazepine suitable for various psychotropic disorders and having less side effects. It is commonly known as quetiapine and is used for the treatment of psychotropic disorders like schizophrenia and acute manic episodes associated with bipolar I disorder.

Quetiapine is an antagonist at multiple neurotransmitter receptors in the brain like serotonin type 1 and 2 (5HT_{1A}, 5HT₂), dopamine type 1 and 2 (D₁, D₂), histamine (H₁) and adrenergic α₁ and α₂ receptors with high affinity for serotonin type-2 (5HT₂) and dopamine type-2 (D₂) receptors.

Sustained release solid oral dosage form of the phenothiazines derivatives is a desired approach in the treatment of psychotropic disorders. The sustained release formulation avoids frequent administration of the medicament while maintaining a uniform and constant release rate of the active pharmaceutical ingredient over an extended period of time.

An effective dissolution profile to maintain effective plasma drug concentration and controlled release rate of medicament after a solid oral drug administration is another reason to formulate sustained release composition of phenothiazine derivatives drugs preferably the dibenzothiazepine compound like quetiapine.

This aim has been attained in the present invention by using a channelizer in the dosage form along with the active pharmaceutical ingredient, rate controlling polymer and optionally other pharmaceutically acceptable excipients. The channelizer acts as a pore forming agent and thereby facilitates release of active pharmaceutical ingredient from the dosage form through the pores and helps the rate controlling polymer to maintain the desired blood plasma concentration of the drug over a prolonged period of time and thereby causing a sustained action of the drug.

### PRIOR ART

WO2005041935 discloses a sustained release solid dosage formulation comprising a matrix, wherein the matrix comprises a therapeutically effective amount of quetiapine or a pharmaceutically acceptable salt thereof, and a wax material.

WO9745124 discloses a sustained release formulation comprising a gelling agent and quetiapine or a pharmaceutically acceptable salt thereof, together with one or more pharmaceutically acceptable excipients. The gelling agent is hydroxypropyl methyl cellulose and pharmaceutically acceptable excipient is selected from the group consisting of microcrystalline cellulose, lactose, magnesium stearate, sodium citrate and povidone, wherein sodium citrate is a pH modifier.

WO0121179 discloses a granule formulation comprising quetiapine or a pharmaceutically acceptable salt thereof and a freely or very water-soluble binder. The granules is dissolved or suspended in a kit comprising an aqueous medium and then used for central nervous system disorders.

WO03039516 discloses a method for improving dissolution of a poorly dispersible medicament like quetiapine, which comprises mixing the poorly dispersible medicament with a floating agent and/or a surfactant and granulating the mixture.

### OBJECT OF THE INVENTION

It is an object of this invention to provide a sustained release orally administrable solid dosage formulation comprising an anti-psychotic drug and pharmaceutically acceptable salt thereof with a channelizer.

It is yet another object of this invention to develop sustained release solid oral dosage formulation of phenothiazine derivative, preferably the heterocyclic analogue of phenothiazine, more preferably the dibenzazepine derivative and most preferably the dibenzothiazepine compound like quetiapine and pharmaceutically acceptable salt thereof.

One more object of this invention is to prepare a sustained release solid oral dosage formulation of phenothiazine derivative and pharmaceutically acceptable salt thereof having an effective dissolution profile.

Another object of the invention is to prepare a sustained release solid oral dosage formulation of phenothiazine derivative and pharmaceutically acceptable salt thereof which provides an effective plasma drug concentration over a prolonged period of time causing a sustained drug action.

Still a further object of the invention is to develop a sustained release solid oral dosage form of phenothiazine derivatives and pharmaceutical acceptable salt thereof which can be used by a patient once a day or twice a day, more preferably once a day.

It is a further object of this invention to provide a process for the preparation of orally administrable sustained release solid oral phenothiazine compound and pharmaceutically acceptable salt thereof with a channelizer.

### SUMMARY OF THE INVENTION

The present invention provides a sustained release solid oral pharmaceutical composition(s) and/or dosage form(s) of phenothiazine derivatives, preferably the heterocyclic analogue of phenothiazine, more preferably the dibenzazepine derivative and most preferably the dibenzothiazepine compound like quetiapine and pharmaceutically acceptable salt thereof consisting of a channelizer which acts as a pore forming agent and facilitates the release of active pharmaceutical ingredient from the dosage form through the pores.

### DETAILED DESCRIPTION OF THE INVENTION

The present invention describes a sustained release solid oral pharmaceutical formulation containing a phenothiazine derivative preferably the heterocyclic analogue of phenothiazine, more preferably the dibenzazepine derivative and most preferably the dibenzothiazepine compound like quetiapine and pharmaceutical acceptable salt thereof as an active ingredient.

The pharmaceutical formulation of the present invention comprises a tablet or capsule as the solid oral dosage form which consist of a core of active pharmaceutical ingredient with a channelizer, rate controlling polymer and optionally one or more pharmaceutically acceptable excipients and a film coating layer surrounding the core.

The formulation of the present invention is preferably based on preparing core by dry granulation, direct compression, wet granulation method or pellet based technology comprising a phenothiazine derivative preferably the dibenzothiazepine compound like quetiapine or its pharmaceutically acceptable salt.

The uncoated granules are compressed into tablets and then film coated or the granules/pellets are film coated and then filled into the capsule.

By the teen sustained release in the present invention it is meant that the therapeutically active medicament is released from the formulation at a controlled rate such that the therapeutically effective amount of active pharmaceutical ingredient is maintained in the blood plasma over an extended period of time to cause the sustained action of the drug.

In a preferred embodiment, the subject formulation comprises core covered by film coating layer.

Core comprises of active pharmaceutical ingredient 30-75%, diluent 10-80%, channelizer 1-5%, rate controlling polymer10-30%, binder 2-5%, glidant 1-3%, lubricant 2-5% and solvent system.

The active pharmaceutical ingredient is selected from the group comprising of phenothiazines derivative. The preferred phenothiazines derivative is the heterocyclic analogue of phenothiazines, more preferably dibenzazepines and most preferably the dibenzothiazepine compound like quetiapine or its pharmaceutically acceptable salt.

Diluent can be selected from the group comprising of but not limited to lactose, sucrose, mannitol, sorbitol, microcrystalline cellulose, calcium phosphates, dextrose, gelatin, acacia, sodium phosphates and the likes.

Channelizer can be selected from the group comprising of but not limited to electrolytes (e.g. sodium chloride and the likes), soluble excipients, osmotic agents, diluents and the likes.

Rate controlling polymers can be selected from the group comprising of but not limited to Pyrollidone derivatives, hydroxy propyl methyl cellulose, HPC, HEC, MC, Vinyl-acetate copolymers, alginate, xanthan gum, guar gum, starch & starch based polymers, poly ethylene oxide, methacrylic acid copolymers, maleic anhydride/methyl vinyl ether copolymers, ethyl cellulose, cellulose acetate, methacrylates, acrylic acid polymers, poly vinyl acetate, wax and the likes.

Binder can be selected from the group comprising of but not limited to polyvinylpyrollidone, starch, methyl cellulose, ethyl cellulose, polyethylene glycol, polyvinyl alcohol, hydroxypropyl cellulose and the likes.

Glidants can be selected from the group comprising of but not limited to talc, sodium stearyl fumerate, magnesium stearate, stearic acid, calcium stearate and the likes.

Lubricant can be selected from the group comprising of but not limited to magnesium stearate, stearic acid, talc, calcium stearate, sodium stearyl fumerate and the likes.

Solvents are used as per the quantity required and can be selected from the group comprising of but not limited to isopropyl alcohol, dichloromethane, methanol, purified water, mixture of likes.

The film coating solution comprises of shellac, zein, hydroxypropyl cellulose, hydroxypropyl methylcellulose, ethyl cellulose, polymethacrylates, polyvinyl acetate phthalate, cellulose acetate phthalate, triacetin, dibutyl sebacate, a mixture of polyethylene glycol, titanium dioxide and hydroxypropyl methylcellulose and the likes. The film coating solution is in the range of 0.2-4%.

The empty capsule in which the coated granules or pellets are filled can be selected from hard gelatin capsule, soft gelatin capsule and the likes.

The dissolution of the active ingredient of the formulation of present invention is in 2 hr 10-45%, in 4 hr 15-60%, in 8 hr 25-75%, in 12 hr 35-80% , in 18 hr not less than 55% and in 24 hr not less than 65%.

The plasma concentration of the active ingredient after the administration of solid oral dosage form of the present invention is above 300 ng/ml between 0.5 to 36 hrs, 300 ng/ml to 3200 ng/ml between 5 to 24 hrs and the area under curve (AUC) is not less than 60% to the two or three immediate release formulations when administered at an interval of 12 hrs. A single or multiple peak plasma concentration appears between 0.5 hrs to 12 hrs after the administration of the formulation of the present invention.

Process for Preparation of Dosage Form:

The manufacturing process of the solid oral dosage form of the present invention involves the following steps:
1. Active pharmaceutical ingredient, diluent, release controlling polymer, and channelizer are dry mixed to get a uniform blend.
2. Granulation of the blend is done by wet granulation, dry granulation or direct compression method. The granules are compressed to form core or the granules are coated with the rate controlling coating solution and filled into the capsule.
3. The compressed core is then film coated using the film coating solution.

Throughout this specification and the appended claims it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

### Example

The present invention has been described by way of example only, and it is to be recognized that modifications thereto falling within the scope and spirit of the appended claims, and which would be obvious to a person skilled in the art based upon the disclosure herein, are also considered to be included within the scope of this invention.

The above said invention can be illustrated by but not limited to following example(s).

TABLE 1
Tablet core:

| | | |
|---|---|---|
| Sr. No. | Ingredients | % Range |

Dry Mixing

| | | |
|---|---|---|
| 1. | Quetiapine Fumarate | 30-75% |
| 2. | H.P.M.C K4M | 10-30% |
| 3. | Lactose | 10-80% |
| 4. | Sodium Chloride | 1-5% |

Granulation

| | | |
|---|---|---|
| 5. | PVP K 30 | 2-5% |
| 6. | Isopropyl Alcohol | Q.S. |
| 7. | Purified water | Q.S. |

Lubrication

| | | |
|---|---|---|
| 8. | Talc | 1-3% |
| 9. | Magnesium Stearate | 2-5% |

Procedure:

Weighed quantity of API, rate controlling polymer, diluent and channelizer are sieved, dry mixed, or granulated with binder solution and then lubricated. The granules are compressed to form tablet using rotary compression machine. The tablets are then coated using film coating solution described in table-2.

TABLE 2
Film Coating Composition:

| Sr. No. | Ingredients | % Range |
|---|---|---|
| 1. | Acryl-eze White | 0.2-4% |
| 2. | Purified Water | Q.S. |

Following examples illustrates the compositions of present invention:

TABLE 3
Composition of core tablet

| | Quantity (mg) | | | | | |
|---|---|---|---|---|---|---|
| Ingredient | Tablet-1 | Tablet-2 | Tablet-3 | Tablet-4 | Tablet-5 | Tablet-6 |
| Intragranular | | | | | | |
| Quetiapine fumerate | 460.54 | 460.54 | 460.54 | 460.54 | 460.54 | 460.54 |
| HPMC K4M | 101.2 | — | 123.84 | 123.84 | 130.72 | 130.72 |
| HPMC K 100 M | — | 101.2 | — | — | — | — |
| Microcrystalline Cellulose | — | — | 73.99 | — | 73.99 | — |
| Lactose | 73.99 | 73.99 | — | 73.99 | — | 73.99 |
| Sodium Chloride | 18 | 18 | 18 | 18 | 18 | 18 |
| Granulation | | | | | | |
| Polyvinyl pyrollidone | 23 | 23 | 23 | 23 | 23 | 23 |
| Isopropyl Alcohol | Q.S. | Q.S. | Q.S. | Q.S. | Q.S. | Q.S. |
| Water | Q.S. | Q.S. | Q.S. | Q.S. | Q.S. | Q.S. |
| Lubrication | | | | | | |
| Talc | 9.85 | 9.85 | 9.85 | 9.85 | 9.85 | 9.85 |
| Magnesium Stearate | 19.69 | 19.69 | 19.69 | 19.69 | 19.69 | 19.69 |
| Total | 706.27 | 706.27 | 728.92 | 728.92 | 735.8 | 735.8 |

Procedure:

Quetiapine fumarate was mixed with microcrystalline cellulose or lactose, sodium chloride, HPMC K4M or HPMC K100M and granulated with polyvinyl pyrollidone dissolved in Isopropyl alcohol or/and purified water mixture. The granules were dried, sized, mixed with magnesium stearate and talc and compressed to form a tablet. The tablets of example 1 to 6 are then film coated using coating solution of table-2.

Dissolution Study:

Tablets of examples 1-6 were tested in dissolution studies in a USP I apparatus in differential pH medium. i.e. 2 hr-0.1 N HCl, 2 hr-Phosphate buffer pH 5.5 & 20 hr-phosphate buffer pH 6.8. The temperature and agitation were set at 37° C.±0.5° C. and 100 rpm, respectively. Aliquots of sample were withdrawn at predetermined time intervals and replaced with an equal amount of fresh media. Samples were processed and suitably analyzed. Dissolution profiles of these tablets are given in Table 4.

TABLE 4
Dissolution profiles of tablets of examples 1-6 as measured
in a USP type I apparatus, at 100 rpm, at a temperature of
37° C. ± 0.5° C. in 900 ml of differential
pH medium i.e. 2 hr-0.1 N HCL, 2 hr-Phosphate buffer
pH 5.5 & 20 hr-Phosphate buffer pH 6.8.

| Time | % Drug release | | | | | |
|---|---|---|---|---|---|---|
| (Hrs) | Tablet 1 | Tablet 2 | Tablet 3 | Tablet 4 | Tablet 5 | Tablet 6 |
| 2 | 36.4 | 33.6 | 30.3 | 33.1 | 30.7 | 34 |
| 4 | 48.9 | 44.1 | 41.3 | 45.4 | 42.2 | 47.3 |
| 8 | 56.1 | 47.7 | 45.2 | 49.5 | 47.1 | 52.9 |
| 12 | 64.3 | 49.4 | 50.7 | 56.9 | 52.6 | 60.7 |
| 18 | 73.7 | 55.8 | 59.1 | 65.6 | 59.6 | 70.8 |
| 24 | 78.9 | 65.8 | 65.7 | 71.8 | 65.9 | 77.9 |

1. A once a day sustained release dosage form comprising a phenothiazine derivative or its pharmaceutically acceptable salt, a channelizer for facilitating release of the phenothiazine derivative or its pharmaceutically acceptable salt from the dosage form through pores that are formed by the channelizer, a release controlling agent and suitable pharmaceutical excipients.

2. The dosage form as claimed in claim 1, wherein the phenothiazine derivative is dibenzothiazepine or its derivative.

3. The dosage form as claimed in claim 2, wherein the dibenzothiazepine derivative is quetiapine.

4. The dosage form as claimed in claim 1, wherein the channelizer is selected from electrolytes, soluble excipients, osmotic agents and diluents.

5. The dosage form as claimed in claim 1, wherein the release controlling agent comprises a hydrophilic, a hydrophobic or a swellable polymers or a mixture thereof.

6. The dosage form as claimed in claim 5, wherein the polymer is selected from the group comprising cellulose derivatives, pyrollidone derivatives, vinyl acetate copolymer, cellulosic acetates, alginates, gums, starch and starch based polymers, methacrylic acid copolymers, polymethacrylates, maleic anhydride/methyl vinyl ether copolymers, wax and poly ethylene oxides.

7. The dosage form as claimed in claim 1, wherein the suitable pharmaceutical excipients are at least one of a diluent, a binder, a glidant, a lubricant, an anti-adhering agents and a colorants.

8. The dosage form as claimed in claims 1, wherein the dosage form comprises a core and a film coating solution surrounding the core.

9. The dosage form as claimed in claim 1, wherein the dissolution profile of the dosage form is 10-45% in 2 hrs, 15-60% in 4 hrs, 25-75% in 8 hrs, 35-80% in 12 hrs, not less than 55% in 18 hrs and not less than 65% in 24 hrs.

10. The dosage form as claimed in claim 1, wherein a plasma concentration of the phenothiazine derivative or its pharmaceutically acceptable salt in a subject after administration of the dosage form is above 300 ng/ml between 0.5 to 36 hrs, and 300 ng/ml to 3200 ng/ml between 5 to 24 hrs, and the dosage form provides an area under curve (AUC) value of the phenothiazine derivative or its pharmaceutically acceptable salt which is not less than 60% of that of the same amount if taken as two or three dosages of an immediate release formulation at an interval of 12 hours.

11. The dosage form as claimed in claim 1, wherein after administration of the dosage form to a subject, a single or multiple peak plasma concentration appears between 0.5 hrs to 12 hrs.

12. A once a day sustained release dosage form comprising quetiapine fumarate in an amount of 55-75% by weight of the dosage form, hydroxy propyl methyl cellulose in an amount of 10-20% by weight of the dosage form, lactose in an amount of 10-25% by weight of the dosage form, sodium chloride in an amount of 1-5% by weight of the dosage form, polyvinyl pyrollidone in an amount of 2-5% by weight of the dosage form, magnesium stearate in an amount of 2-5% by weight of the dosage form and talc in an amount of 1-3% by weight of the dosage form.

13. The dosage form as claimed in claim 12, wherein a dissolution profile of the dosage form is 35-45% in 2 hours, 45-55% in 4 hours, 60-70% in 8 hours, 65-75% in 12 hours, 75-85% in 18 hours and not less than 80% in 24 hours.

14. The dosage form as claimed in claims 1, wherein the dosage form comprises granules prepared by a dry granulation method or wet granulation method and compressed into a tablet, which is then film coated.

15. The dosage form as claimed in claims 1, wherein the dosage form comprises granules, pellets or coated pellets that are directly filled inside a capsule.

16. (canceled)

17. The dosage form as claimed in claim 1, wherein dosage form comprises pellets that are film coated and compressed into a tablet with further optional coating.

18. The dosage form as claimed in claim 8, wherein the core comprises the phenothiazine derivative or its pharmaceutically acceptable salt in an amount of 30-75% by weight of the dosage form, diluent in an amount of 10-80% by weight of the dosage form, the channelizer in an amount of 1-5% by weight of the dosage form, the release controlling agent in an amount of 10-30% by weight of the dosage form, binder in an amount of 2-5% by weight of the dosage form, glidant in an amount of 1-3% by weight of the dosage form, and lubricant in an amount of 2-5% by weight of the dosage form, and the film coating solution makes up 0.2-4% by weight of the dosage form.

19. The dosage form as claimed in claim 12, wherein the dosage form comprises granules that are prepared by a dry granulation method or wet granulation method and compressed into a tablet, which is then film coated.

20. The dosage form as claimed in claim 12, wherein the dosage form comprises granules, pellets or coated pellets that are directly filled inside a capsule.

21. A once a day sustained release dosage form comprising an active pharmaceutical ingredient in an amount of 30-75% by weight of the dosage form, diluent in an amount of 10-80% by weight of the dosage form, pore forming agent in an amount of 1-5% by weight of the dosage form, release controlling agent in an amount of 10-30% by weight of the dosage form, binder in an amount of 2-5% by weight of the dosage form, glidant in an amount of 1-3% by weight of the dosage form, lubricant in an amount of 2-5% by weight of the dosage form, and film coating solution in an amount of 0.2-4% by weight of the dosage form.

22. The dosage form as claimed in claim 21, wherein the dissolution profile of the dosage form is 10-45% in 2 hrs, 15-60% in 4 hrs, 25-75% in 8 hrs, 35-80% in 12 hrs, not less than 55% in 18 hrs and not less than 65% in 24 hrs.

23. The dosage form as claimed in claim 21, wherein a plasma concentration of the active pharmaceutical ingredient in a subject after administration of the dosage form is above 300 ng/ml between 0.5 to 36 hrs, and 300 ng/ml to 3200 ng/ml between 5 to 24 hrs and the dosage form provides an area under curve (AUC) value of the active pharmaceutical ingredient which is not less than 60% of that of the same amount if taken as two or three dosages of an immediate release formulation at an interval of 12 hours.

24. The dosage form as claimed in claim 21, wherein after administration of the dosage form to a subject, a single or multiple peak plasma concentration appears between 0.5 hrs to 12 hrs.

25. A treatment method comprising using the dosage form of claim 1 to treat a psychotropic disorder.

26. A treatment method comprising using the dosage form of claim 12 to treat a psychotropic disorder.

27. A treatment method comprising using the dosage form of claim 21 to treat a psychotropic disorder.

28. A method of treating a psychotropic disorder comprising administering to a mammalian patient in need of a treatment a therapeutically effective amount of the dosage form of claim 1.

29. A method of treating a psychotropic disorder comprising administering to a mammalian patient in need of a treatment a therapeutically effective amount of the dosage form of claim 12.

30. A method of treating a psychotropic disorder comprising administering to a mammalian patient in need of a treatment a therapeutically effective amount of the dosage form of claim 21.
